(19) United States
(12) Patent Application Publication
(10) Pub. No.: US 2024/0274242 A1
(43) Pub. Date: Aug. 15, 2024

(54) METHOD AND SYSTEM FOR NORMALIZATION OF SEAWATER SAMPLES

(71) Applicant: ACWA POWER Company, Riyadh (SA)

(72) Inventors: Thomas ALTMANN, Dubai (AE); Ahmed Yousry Ahmed MAHMOUD, Dubai (AE); Muhammad Ghifari RIDWAN, Dubai (AE); Ratul DAS, Makkah (SA)

(73) Assignee: ACWA POWER Company, Riyadh (SA)

(21) Appl. No.: 18/438,032

(22) Filed: Feb. 9, 2024

Related U.S. Application Data
(60) Provisional application No. 63/444,780, filed on Feb. 10, 2023.

(51) Int. Cl. G16C 20/20; G01N 33/18; G16C 20/70

(52) U.S. Cl. CPC G16C 20/20; G01N 33/18; G16C 20/70

(57) ABSTRACT

A system and method retrieves liquid stream data associated with a first liquid stream that includes a set of chemical compounds. and a set of parameters. The system also retrieves reference data that is associated with a reference liquid stream, and includes a set of reference parameters. The system compares at least a first parameter of the set of liquid-stream parameters with a corresponding reference parameter of the set of reference parameters. The system also controls an output of the first liquid stream based on the comparison of parameters.

### CROSS-REFERENCE TO RELATED APPLICATION

This application claims priority to U.S. Provisional Patent Application Ser. No. 63/444,780, filed Feb. 10, 2023, and entitled “METHOD AND SYSTEM FOR NORMALIZATION OF SEAWATER SAMPLES”, the disclosure of which is incorporated herein by reference.

### TECHNICAL FIELD

This disclosure relates to the field of liquid treatment. More specifically, the present disclosure relates to a seawater reverse osmosis (SWRO) system for normalization of seawater sample.

### BACKGROUND

To meet increasing global demand for freshwater, seawater reverse osmosis (SWRO) is being widely used technology to produce freshwater from seawater. The SWRO process takes place in a seawater reverse osmosis (SWRO) plant. Such SWRO plants include multiple components such as pumps, membranes, valves, energy recovery devices, and the like. The SWRO process can be complex, counterintuitive, as well as energy-intensive. Therefore, to minimize the specific energy consumption (SEC) of the SWRO process, it is of utmost importance that the performance of the SWRO plant is monitored in real-time.

Furthermore, due to periodic changes in seawater quality and weather conditions, the operating parameters in a seawater reverse osmosis (SWRO) plant need continuous tweaking to minimize energy consumption and optimize the use of chemicals such that produced water meets the pre-set quality requirements. Therefore, there is a requirement for a performance monitoring and optimization tool for SWRO plants.

### BRIEF SUMMARY

A system and a method are provided herein that focuses on the optimization and design of the seawater reverse osmosis (SWRO) process with the facilitation of the seawater sample normalization algorithm.

In one aspect, a system for normalization of seawater samples is provided. The system may include a memory that may be configured to execute the computer executable instructions that may cause the one or more processors to retrieve liquid stream data associated with a first liquid stream including a set of chemical compounds. The liquid stream data may include a set of parameters. The one or more processors may be further configured to retrieve reference data associated with a reference liquid stream. The reference data may include a set of reference parameters. The one or more processors may be further configured to compare at least a first parameter of the set of parameters with a corresponding reference parameter of the set of reference parameters. Further, the one or more processors may be configured to control an output of the first liquid stream based on the comparison.

In an embodiment, the set of parameters is associated with at least one of a chemical composition of each of the set of chemical compounds in the first liquid stream, a potential of hydrogen (pH) of the first liquid stream, a total dissolved solid (TDS) of the first liquid stream, a charge balance of the set of chemical compounds in the first liquid stream, one or more water quality parameters of the first liquid stream, and a chloride ion ratio of the first liquid stream.

In an embodiment, the set of reference parameters is associated with at least one of a chemical composition for each of the set of chemical compounds in the reference liquid stream, a pH of the reference liquid stream, a TDS of the reference liquid stream, a charge balance of the set of chemical compounds in the reference liquid stream, one or more water quality parameters of the reference liquid stream, and a chloride ion ratio of the reference liquid stream.

In an embodiment, the one or more processors may be further configured to determine whether chemical composition data for each of the set of chemical compounds of the first liquid stream may be within a first threshold range. The first threshold range may include at least a minimum chemical composition value for a corresponding chemical compound and a maximum chemical composition value for the corresponding chemical compound. The minimum chemical composition value and the maximum chemical composition value may be determined based on chemical composition data for each of the set of chemical compounds of the reference liquid stream.

In an embodiment, the one or more processors may be further configured to control the output of the first liquid stream based on a determination that the chemical composition data for each of the set of chemical compounds of the first liquid stream is within the first threshold range. The output of the first liquid stream corresponds to an adjustment of the chemical composition data for at least a first chemical compound of the set of chemical compounds of the first liquid stream iteratively until a TDS data of the first liquid stream is within a second threshold range.

In an embodiment, the second threshold range may include at least a minimum TDS value of the first liquid stream and a maximum TDS value of the first liquid stream. The minimum TDS value and the maximum TDS value may be determined based on TDS data of the reference liquid stream.

In an embodiment, the one or more processors may be further configured to control the output of the first liquid stream based on a determination that the chemical composition data for each of the set of chemical compounds of the first liquid stream may be less than the minimum chemical composition value for a corresponding chemical compound, or greater than the maximum chemical composition value for the corresponding chemical compound. The output of the first liquid stream corresponds to an adjustment of the chemical composition data for a second chemical compound of the set of chemical compounds of the first liquid stream iteratively until the pH data of the first liquid stream may be within a third threshold range.

In an embodiment, the third threshold range may be at least a minimum pH value of the first liquid stream and a maximum pH value of the first liquid stream. The minimum pH value and the maximum pH value may be determined based on the pH data of the reference liquid stream.

In another aspect, the method for normalization of seawater sample is provided. The method may include retrieving liquid stream data associated with a first liquid stream including a set of chemical compounds. The liquid stream data may include a set of parameters. The method may further include retrieving reference data associated with a reference liquid stream. The reference data may include a set of reference parameters. The method may further include comparing at least a first parameter of the set of parameters with a corresponding reference parameter of the set of reference parameters. Further, the method may include controlling an output of the first liquid stream based on the comparison.

In a method embodiment, the set of parameters may be associated with at least one of: a chemical composition or each of the set of chemical compounds in the first liquid stream, a potential of hydrogen (pH) of the first liquid stream, a total dissolved solid (TDS) of the first liquid stream, a charge balance of the set of chemical compounds in the first liquid stream, one or more water quality parameters of the first liquid stream, and a chloride ion ratio of the first liquid stream.

In a method embodiment, the method may further include determining whether chemical composition data for each of the set of chemical compounds of the first liquid stream is within a first threshold range. The first threshold range may include at least a minimum chemical composition value for a corresponding chemical compound and a maximum chemical composition value for the corresponding chemical compound. The minimum chemical composition value and the maximum chemical composition value may be determined based on chemical composition data for each of the set of chemical compounds of the reference liquid stream.

In a method embodiment, the method may further include controlling the output of the first liquid stream based on a determination the chemical composition data for each of the set of chemical compounds of the first liquid stream is within the first threshold range. The output of the first liquid stream corresponds to an adjustment of the chemical composition data for at least a first chemical compound of the set of chemical compounds of the first liquid stream iteratively until the TDS data of the first liquid stream may be within a second threshold range.

In a method embodiment, the second threshold range may include at least a minimum TDS value of the first liquid stream and a maximum TDS value of the first liquid stream. The minimum TDS value and the maximum TDS value may be determined based on TDS data of the reference liquid stream.

In a method embodiment, the method further includes controlling the output of the first liquid stream based on a determination that the chemical composition data for each of the set of chemical compounds of the first liquid stream is less than the minimum chemical composition value for a corresponding chemical compound, or greater than the maximum chemical composition value for the corresponding chemical compound. The output of the first liquid stream corresponds to an adjustment of the chemical composition data for a second chemical compound of the set of chemical compounds of the first liquid stream iteratively until the pH data of the first liquid stream may be within a third threshold range.

In a method embodiment, the third threshold range may include at least a minimum pH value of the first liquid stream and a maximum pH value of the first liquid stream. The minimum pH value and the maximum pH value may be determined based on the pH data of the reference liquid stream.

In yet another aspect, a non-transitory computer-readable storage medium carrying one or more sequences of one or more instructions which, when executed by at least one processor, cause the system to perform operations comprising retrieving liquid stream data associated with a first liquid stream may include a set of chemical compounds. The liquid stream data may include a set of parameters. The operations may further include retrieving reference data associated with a reference liquid stream. The reference data may include a set of reference parameters. The operation may further include comparing at least a first parameter of the set of parameters with a corresponding reference parameter of the set of reference parameters. Further, the operations may include controlling an output of the first liquid stream based on the comparison.

In an embodiment, the set of parameters may be associated with at least one of a chemical composition or each of the set of chemical compounds in the first liquid stream, a potential of hydrogen (pH) of the first liquid stream, a total dissolved solids (TDS) of the first liquid stream, a charge balance of the set of chemical compounds in the first liquid stream, one or more water quality parameters of the first liquid stream, and a chloride ion ratio of the first liquid stream.

In an embodiment, the set of reference parameters may be associated with at least one of a chemical composition for each of the set of chemical compounds in a reference liquid stream, a pH of the reference liquid stream, a TDS of the reference liquid stream, a charge balance of the set of chemical compounds in the reference liquid stream, one or more water quality parameters of the reference liquid stream, and a chloride ion ratio of the reference liquid stream.

In an embodiment, the operations may further include determining whether chemical composition data for each of the set of chemical compounds of the first liquid stream is within a first threshold range. The first threshold range may include at least a minimum chemical composition value for a corresponding chemical compound and a maximum chemical composition value for the corresponding chemical compound. The minimum chemical composition value and the maximum chemical composition value may be determined based on chemical composition data for each of the set of chemical compounds of the reference liquid stream.

### BRIEF DESCRIPTION OF THE DRAWINGS

Having thus described example embodiments of the invention in general terms, reference will now be made to the accompanying drawings, which are not necessarily drawn to scale, and wherein:

FIG. 1 illustrates a network environment in which the system for the normalization of seawater sample is implemented, in accordance with an embodiment of the disclosure;

FIG. 2 illustrates a block diagram of the system of FIG. 1, in accordance with an embodiment of the disclosure;

FIG. 3 illustrates exemplary operations for the seawater sample normalization system, in accordance with an embodiment of the disclosure;

FIG. 4 illustrates exemplary operations for adjustment of the TDS for normalization of seawater sample, in accordance with an embodiment of the disclosure;

FIG. 5 illustrates exemplary operations for adjustment of the pH for normalization of seawater sample, in accordance with an embodiment of the disclosure; and

FIG. 6 is a flowchart that illustrates an exemplary method for the normalization of seawater sample, in accordance with an embodiment of the disclosure.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

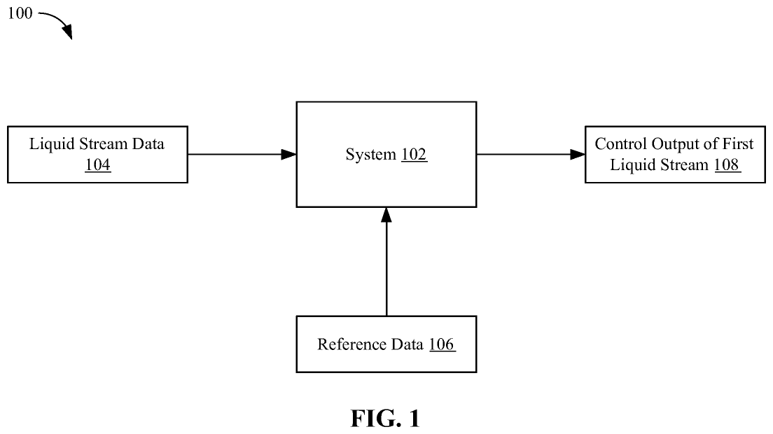

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect. Turning now to FIG. 1-FIG. 6, a brief description concerning the various components of the present disclosure will now be briefly discussed. Reference will be made to the figures showing various embodiments of the system for the normalization of seawater.

FIG. 1 illustrates a network environment in which the system for normalization of seawater sample is implemented, in accordance with an embodiment of the disclosure. With reference to FIG. 1, there is shown a network environment 100. The network environment 100 may include a system 102 for normalization of seawater.

In an embodiment, the system 102 may correspond to a seawater normalization system that may have the capability of neutralizing the charge of an unbalanced seawater sample and correcting its ion-to-TDS ratio thereby, providing better inputs for designing a seawater reverse osmosis process. The system 102 may be based on the comparison of the liquid stream data 104 to reference data 106 to perform accuracy checks. The system 102 may enhance data acceptability, minimize deviations, and standardize inaccurate seawater analyses for utilization in SWRO plant designs. The system 102 may be a simulation system for simulating the operation of an entire SWRO plant. The seawater sample standardization, by error elimination, may be the basis for obtaining the best possible operating parameters in SWRO at minimum energy consumption. The system 102 may be, for example, but not limited to, a reverse osmosis simulation system, a multi-stage flash distillation simulation system, a multiple effect distillation simulation system, and an electrodialysis reversal simulation system.

In operation, the system 102 may be configured to retrieve the liquid stream data 104. The liquid stream data 104 may be associated with the first liquid stream. In an embodiment, the first liquid stream may correspond to an unbalanced seawater. The liquid stream data 104 may include the set of parameters. The set of parameters may be associated with at least one of: a chemical composition of each of the set of chemical compounds in the first liquid stream, a potential of hydrogen (pH) of the first liquid stream, a total dissolved solid (TDS) of the first liquid stream, a charge balance of the set of chemical compounds in the first liquid stream, one or more water quality parameters of the first liquid stream, and a chloride ion ratio of the first liquid stream.

In an embodiment, the system 102 may be configured to determine the chemical composition of each of the set of chemical compounds in the first liquid stream. The chemical composition of the set of chemical compounds may correspond to the concentration of set of chemical ions present in the first liquid stream. The set of chemical compounds that may be present in the first liquid stream may correspond to set of chemical ions present in the first liquid stream, for example, but not limited to, chloride (Cl⁻), sodium (Na⁺), sulfate (SO²⁻₄), magnesium (Mg²⁺), calcium (Ca²⁺), and potassium (K⁺).

In another embodiment, the system 102 may retrieve the reference data 106. The reference data 106 may be associated with the reference liquid stream. The reference liquid stream may be the sample of seawater. Such a sample of seawater may be obtained from sources, for example, but not limited to, a storage tank, a seawater intake pipe, and a well. In such a scenario, the reference data 106 may correspond to sample seawater data that may include standard seawater parameters. The reference data 106 may include a set of reference parameters. The set of reference parameters may be associated with at least one of: a chemical composition for each of the set of chemical compounds in the reference liquid stream, a pH of the reference liquid stream, a TDS of the reference liquid stream, a charge balance of the set of chemical compounds in the reference liquid stream, one or more water quality parameters of the reference liquid stream, and a chloride ion ratio of the reference liquid stream.

In an embodiment, the system 102 may be configured to compare at least the first parameter of the set of parameters with the corresponding reference parameter of the set of reference parameters. In an exemplary embodiment, the first parameter of the set of parameters may be the set of chemical compounds of the first liquid stream. The system 102 may determine whether chemical composition data for each of the set of chemical compounds of the first liquid stream 108 may be within a first threshold range. The first threshold range may include at least a minimum chemical composition value for a corresponding chemical compound and a maximum chemical composition value for the corresponding chemical compound. Further, the minimum chemical composition value and the maximum chemical composition value may be determined based on chemical composition data for each of the set of chemical compounds of the reference liquid stream.

By way of an example, a first chemical compound of the set of chemical compounds of the first liquid stream 108 may correspond to sodium. The system 102 may be configured to determine whether the chemical composition data of the sodium in the first liquid stream 108 is within the first threshold range. The system 102 may determine whether the chemical composition data of the sodium in the first liquid stream 108 lies between the minimum chemical composition value and the maximum chemical composition value. The minimum chemical composition value and the maximum chemical composition value may be determined based on the chemical composition data for sodium that may be present in the reference liquid stream.

In accordance with the present example, the chemical composition data for the sodium that may be present in the reference liquid stream may be, for example, 10.6 g/Kg. The minimum chemical composition value associated with the first threshold range may be −5% of the sodium ions that may be present in the reference liquid stream, for example, 10.07 g/Kg, and the maximum chemical composition value associated with the first threshold range may be +5% the sodium ions for example, 11.13 g/Kg, that may be present in the reference liquid stream.

In a case, where the determined chemical composition data of the sodium ions may be less than the minimum chemical composition value associated with the first threshold range of the sodium ions that may be present in the reference liquid stream, the system 102 may be configured to modify the chemical composition of the sodium ions in the first liquid stream 108. Further, the system 102 may be configured to extract the sodium ions from the reference liquid stream and add the extracted sodium ions from the reference liquid stream to the first liquid stream 108 till the chemical composition data of the sodium ions in the first liquid stream 108 may be within the first threshold range.

In another case, where the determined chemical composition data of the sodium ions may be greater than the maximum chemical composition value associated with the first threshold range of the sodium ions that may be present in the reference liquid stream, the system 102 may be configured to extract the sodium ions from the first liquid stream 108 until the chemical composition data of the sodium ions in the first liquid stream 108 may be within the first threshold range.

In an embodiment, the system 102 may perform the steps in the above-mentioned example for adjusting the chemical composition data for each of the chemical compounds of the first liquid stream 108.

In an embodiment, upon adjusting the chemical composition data of the chemical compounds of the set of chemical compounds within the first liquid stream 108, the system 102 may output the first liquid stream 108. In an exemplary embodiment, the first liquid stream 108 that the system 102 may output may be used as an input for the reverse osmosis simulation process. The chemical composition data of the at least first chemical compound of the set of chemical compounds of the first liquid stream 108 may be further adjusted until the TDS data of the first liquid stream 108 may be within a second threshold range.

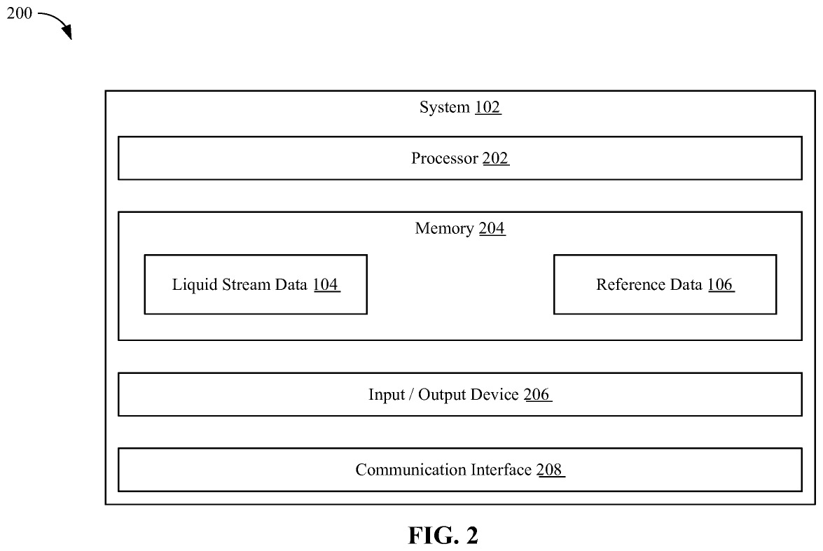

FIG. 2 illustrates a block diagram 200 of the system of FIG. 1, in accordance with an embodiment of the disclosure. FIG. 2 is explained in conjunction with FIG. 1. In FIG. 2, there is shown the block diagram 200 of the system 102. The system 102 may include at least one processor (referred to as a processor 202, hereinafter), at least one non-transitory memory (referred to as a memory 204, hereinafter), an input/output (I/O) interface 206, and a communication interface 208. The memory 204 may further include the liquid stream data 104 and the pre-defined reference data 106. The processor 202 may be connected to the memory 204, the I/O interface 206, and the communication interface 208 through one or more wired or wireless connections. Although in FIG. 2, it is shown that the system 102 includes the processor 202, the memory 204, the I/O interface 206, and the communication interface 208 however, the disclosure may not be so limiting and the system 102 may include fewer or more components to perform the same or other functions of the system 102.

The processor 202 of the system 102 may be configured to perform one or more operations associated with the seawater reverse osmosis. The processor 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application-specific integrated circuit), an FPGA (field programmable gate array), a microcontroller unit (MCU), a hardware accelerator, a special-purpose computer chip, or the like. As such, in some embodiments, the processor 202 may include one or more processing cores configured to perform independently. A multi-core processor may enable multiprocessing within a single physical package. Additionally, or alternatively, the processor 202 may include one or more processors configured in tandem via the bus to enable independent execution of instructions, pipelining, and/or multithreading. Additionally, or alternatively, the processor 202 may include one or more processors capable of processing large volumes of workloads and operations to provide support for big data analysis. In an example embodiment, the processor 202 may be in communication with the memory 204 via a bus for passing information among components of the system 102.

For example, when the processor 202 may be embodied as an executor of software instructions, the instructions may specifically configure the processor 202 to perform the algorithms and/or operations described herein when the instructions are executed. However, in some cases, the processor 202 may be a processor-specific device (for example, a mobile terminal or a fixed computing device) configured to employ an embodiment of the present disclosure by further configuration of the processor 202 by instructions for performing the algorithms and/or operations described herein. The processor 202 may include, among other things, a clock, an arithmetic logic unit (ALU), and logic gates configured to support the operation of the processor 202. The communication network may be accessed using the communication interface 208 of the system 102. The communication interface 208 may provide an interface for accessing various features and data stored in the system 102.

The memory 204 may be non-transitory and may include, for example, one or more volatile and/or non-volatile memories. In other words, for example, the memory 204 may be an electronic storage device (for example, a computer readable storage medium) comprising gates configured to store data (for example, bits) that may be retrievable by a machine (for example, a computing device like the processor 202). The memory 204 may be configured to store information, data, content, applications, instructions, or the like, for enabling the system 102 to carry out various functions in accordance with an example embodiment of the present disclosure. For example, the memory 204 may be configured to buffer input data for processing by the processor 202. As exemplified in FIG. 2, the memory 204 may be configured to store instructions for execution by the processor 202. As such, whether configured by hardware or software methods, or by a combination thereof, the processor 202 may represent an entity (for example, physically embodied in circuitry) capable of performing operations according to an embodiment of the present disclosure while configured accordingly. Thus, for example, when the processor 202 is embodied as an Application Specific Integrated Circuit (ASIC), Field Programmable Gate Array (FPGA), or the like, the processor 202 may be specifically configured hardware for conducting the operations described herein. In an embodiment, memory may be configured to store the liquid stream data 104 and the reference data 106.

In an embodiment, the processor 202 may be configured to retrieve the liquid stream data 104 and store the liquid stream data 104 in the memory 204. The liquid stream data 104 may be associated with the first liquid stream 108 which may be the unbalanced water. In another embodiment, the processor 202 may be configured to retrieve the reference data 106 and store the pre-defined reference data 106 in the memory 204. The reference data 106 may be associated with the reference liquid stream that may correspond to the sample seawater.

In some example embodiments, the I/O interface 206 may communicate with the system 102 and display the input and/or output of the system 102. As such, the I/O interface 206 may include a display and, in some embodiments, may also include a keyboard, a mouse, a touch screen, touch areas, soft keys, or other input/output mechanisms. In one embodiment, the system 102 may include a user interface circuitry configured to control at least some functions of one or more I/O interface elements such as a display and, in some embodiments, a plurality of speakers, a ringer, one or more microphones and/or the like. The processor 202 and/or I/O interface 206 circuitry including the processor 202 may be configured to control one or more functions of one or more I/O interface 206 elements through computer program instructions (for example, software and/or firmware) stored on a memory 204 accessible to the processor 202.

The communication interface 208 may include the input interface and output interface for supporting communications to and from the system 102 or any other component with which the system 102 may communicate. The communication interface 208 may be any means such as a device or circuitry embodied in either hardware or a combination of hardware and software that is configured to receive and/or transmit data to/from a communications device in communication with the system 102. In this regard, the communication interface 208 may include, for example, an antenna (or multiple antennae) and supporting hardware and/or software for enabling communications with a wireless communication network. Additionally, or alternatively, the communication interface 208 may include the circuitry for interacting with the antenna(s) to cause transmission of signals via the antenna(s) or to handle receipt of signals received via the antenna(s). In some environments, the communication interface 208 may alternatively or additionally support wired communication. As such, for example, the communication interface 208 may include a communication modem and/or other hardware and/or software for supporting communication via cable, digital subscriber line (DSL), universal serial bus (USB), or other mechanisms.

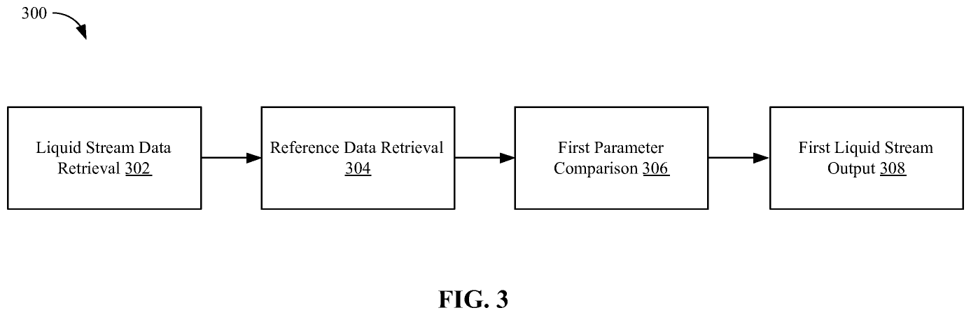

FIG. 3 illustrates exemplary operations for normalization of seawater sample, in accordance with an embodiment of the disclosure. FIG. 3 is explained in conjunction with FIG. 1 and FIG. 2. In FIG. 3 there is shown a block diagram 300 of the system 102 for seawater normalization. The system 102 may include a liquid stream data retrieval 302, a reference data retrieval 304, a first parameter comparison 306, and a first liquid stream output 308.

At 302, the processor 202 may be configured to perform the liquid stream data retrieval operation. The liquid stream data 104 may correspond to the data of the first liquid stream 108. The first liquid stream may correspond to the unbalanced seawater. The first liquid stream 108 may include a set of chemical compounds. The liquid stream data 104 may include the set of parameters.

In an embodiment, the system 102 may retrieve the liquid stream data 104 and further process it to neutralize the charge of the first liquid stream 108 and correct the ion to TDS ratio of the first liquid stream 108 to provide better input for designing the seawater reverse osmosis process.

In an embodiment, the set of parameters associated with the liquid stream data 104 may include at least one of the chemical compositions for each of the set of chemical compounds in the first liquid stream 108. In an example, the chemical composition for each of the set of chemical compounds in the first liquid stream may correspond to a concentration of each of the set of chemical compounds. The set of chemical compounds may include, for example, but not limited to, sulphate, magnesium, calcium, and potassium. Further, the set of parameters may include, for example, but not limited to, the potential of hydrogen (pH) of the first liquid stream, the total dissolved solids (TDS) of the first liquid stream, the charge balance of the set of chemical compounds in the first liquid stream, the one or more water quality parameters of the first liquid stream, or the chloride ion ratio of the first liquid stream.

In an example, the one or more water quality parameters of the first liquid stream may include, but are not limited to at least one of: the physical parameters, chemical parameters, and biological parameters. The physical parameters may include, but are not limited to, colour, taste, odour, temperature, turbidity, solids, and electrical conductivity. The chemical parameters may include, include, but are not limited to pH, acidity, alkalinity, chlorine, hardness, dissolved oxygen, and biological oxygen. The biological parameters, may include, but are not limited to bacteria, algae, and viruses.

At 304, the processor 202 may be configured to perform the reference data 106 retrieval operation. The reference data 106 may be associated with the reference liquid stream. The reference data 106 may correspond to the reference seawater sample data that may be obtained either from standard seawater ratios or from an average balanced sample for the same location.

The reference data 106 may include at least one of the chemical composition for each of the set of chemical compounds in the reference liquid stream, the pH of the reference liquid stream, the TDS of the reference liquid stream, the charge balance of the set of chemical compounds in the reference liquid stream, the one or more water quality parameters of the reference liquid stream, or the chloride ion ratio of the reference liquid stream.

At 306, the processor 202 may be configured to perform the first parameter comparison operation. In the first parameter comparison operation, the processor 202 may be configured to determine whether the chemical composition data for each of the set of chemical compounds of the first liquid stream is within their corresponding threshold range.

In an exemplary embodiment, the processor 202 may compare each chemical compound in the set of chemical compounds of the first liquid stream, and check deviations from each of the respective chemical compounds of the set of chemical compounds associated with the reference liquid stream. Based on the comparison, the chemical compounds of the first liquid stream with deviations of more or less than 5% from the respective chemical compounds of the reference liquid stream may be corrected by reducing the deviation within a first threshold range for example, to a maximum or minimum of 5% deviation of the corresponding chemical compound.

At 308, the processor 202 may be configured to perform the first liquid stream 108 output operation. The processor 202 may control the output of the first liquid stream 108. The output of the first liquid stream 108 may be based on the determination that the chemical composition data for each of the set of chemical compounds of the first liquid stream 108 may be within the first threshold range, for example, a maximum of 5% deviation of the reference liquid stream. In such an embodiment, the output corresponds to a storage of the first liquid stream. The output may correspond to sending the normalized water to water tanks.

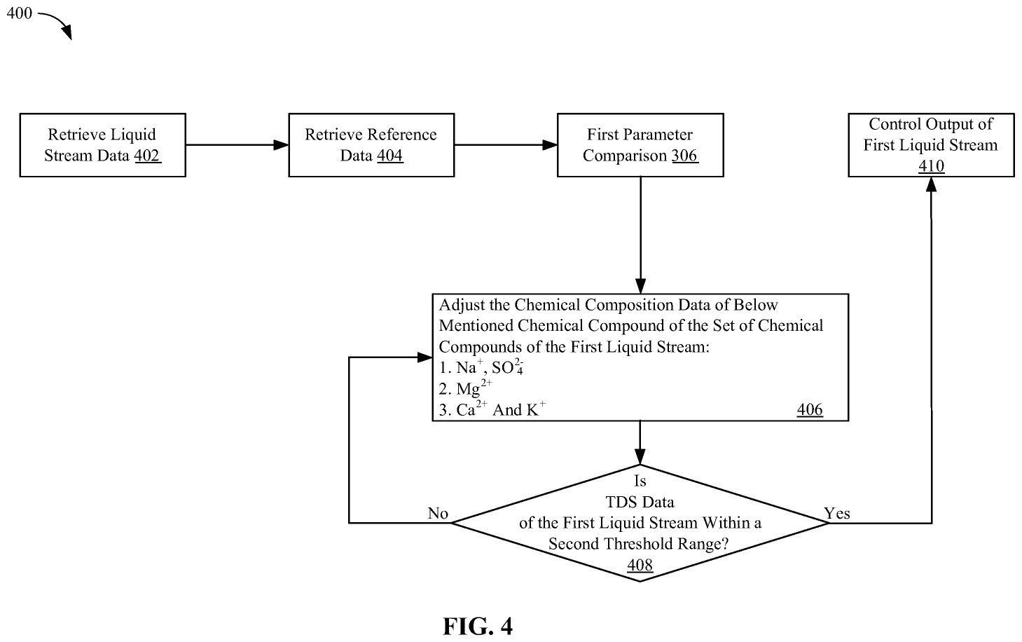

In a case, where the chemical composition data of the each of chemical compounds of the first liquid stream 108 may be beyond the first threshold range, the processor 202 may control the output of the first liquid stream 108. The output of the first liquid stream 108 may further correspond to adjusting the chemical composition data of at least the first chemical compound of the first set of chemical compounds of the first liquid stream. The adjustment may take place iteratively until the TDS data of the first liquid stream 108 is within the second threshold range. The details of the second threshold range are provided in FIG. 4.

FIG. 4 illustrates exemplary operations for adjustment of the TDS for normalization of seawater sample, in accordance with an embodiment of the disclosure. FIG. 4 is explained in conjunction with FIG. 1, FIG. 2, and FIG. 3. In FIG. 4 there is shown a block diagram 400 of the system 102 for normalization of seawater.

At 402, the processor 202 may be configured to retrieve liquid stream data 104. The liquid stream data 104 may be associated with the first liquid stream 108. Further, the liquid stream data 104 may include a set of parameters. The first liquid stream 108 may correspond to the unbalanced seawater. The unbalanced seawater may be seawater that may have an irregular or non-ideal composition in terms of chemical and mineral content. The first liquid stream 108 may have deviations from a reference or standard composition. The deviations may be caused due to factors that may be, for example, but not limited to, seasonal changes, environmental influences, or errors in sample collection and analysis.

In an embodiment, the liquid stream data 104 corresponds to the data of the first liquid stream. The liquid stream data 104 may include the set of parameters and each of the set of parameters may include, for example, but not limited to, at least one of a chemical composition of each of the set of chemical compounds in the first liquid stream, a potential of hydrogen (pH) of the first liquid stream, a total dissolved solids (TDS), of the first liquid stream, a charge balance of the set of chemical compounds in the first liquid stream, one or more water quality parameters of the first liquid stream 108 an a chloride ion ratio of the first liquid stream.

In an exemplary embodiment, the set of chemical compounds of the first liquid stream 108 may include, but is not limited to, sodium, chloride, magnesium, sulphate, calcium, potassium, strontium, bromide, and boron.

In an exemplary embodiment, the TDS of the first liquid stream 108 may be the total amount of solids that may be dissolved in the first liquid stream, including soluble hydrogen carbonate ions, chloride salts, sulphates, calcium, magnesium, sodium, potassium, volatile solids, and non-volatile solids. In an exemplary embodiment, the system 102 may be configured to determine the sum of the chemical composition of each chemical compound of the set of chemical compounds of the first liquid stream 108. The determined sum is the TDS of the first liquid stream 108.

In another exemplary embodiment, the system 102 may be configured to determine the TDS of the first liquid stream 108 by using the Electrical Conductivity (EC) of the first liquid stream 108. The EC of the first liquid stream may be a measure of the ability of water to pass electrical flow. The EC of the first liquid stream 108 may be determined by a portable water quality checker device that may be integrated with the system 102. The system 102 may determine the TDS of the first liquid stream 108 based on the equation given below:

$$TDS ( mg / L ) = k \times EC ( μ S / cm )$$

where,
TDS is the TDS of the first liquid stream;
k is the constant of proportionality; and
EC is the electrical conductivity of the first liquid stream 108.

It may be understood by one of ordinary skill in the art that the equation mentioned above may be correspond to a general calculation of the TDS, the system 102 may calculate the TDS using other mathematical equations, without deviating from the scope of the present disclosure.

At 404, the processor 202 may be configured to retrieve reference data 106. The reference data 106 may be associated with the reference liquid stream. Further, the reference data 106 may include a set of reference parameters. In an exemplary embodiment, the reference liquid stream may be, for example, but not limited to, a reference seawater sample.

In an embodiment, the reference seawater sample may be obtained from standard seawater ratios. The standard seawater ratios may be the composition of major ions that may be present in the seawater. The standard seawater ratios may be important for understanding the chemical composition of the chemical compounds in the reference liquid stream. In another embodiment, the reference seawater sample may be obtained from an average sample that may represent the first liquid stream sample from the same location. The standard seawater ratios may be accurately measured by, for example, a Conductivity Temperature and Depth (CTD) device. The CTD device may be integrated with the system 102. Further, the CTD device may be an oceanographic instrument that may be used to measure the conductivity, temperature, and depth of the seawater. The measurements made by the CTD device may be valuable data for understanding the physical and chemical properties of the seawater.

In an embodiment, upon the retrieval of the liquid stream data 104 and the reference data 106, the processor 202 may compare the first parameter of the set of parameters associated with the first liquid stream 108 with the first parameter of the set of reference parameters. Based on the comparison the processor 202 may output the first liquid stream.

In an embodiment, upon retrieving the liquid stream data 104 and the reference data 106, the system 102 may be configured to compare at least a first parameter of the set of parameters with a corresponding reference parameter of the set of reference parameters. For example, in a case where the first parameter of the set of parameters may be the set of chemical compounds associated with the first liquid stream, the system 102 may be configured to compare the each of the set of chemical compounds with the respective chemical compound of the set of chemical compounds associated with the reference liquid stream.

In an exemplary embodiment, based on the comparison, the system 102 may provide a complete seawater analysis by adjusting the chemical composition data for each of the set of chemical compounds of the first liquid stream 108. The processor 202 may control the addition of the chemical compounds of the set of chemical compounds in the first liquid stream 108 that may not be within the first threshold range with equivalent chemical compounds of the set of chemical compounds from reference liquid stream compositions. The reference liquid stream compositions can be either standard seawater or an average sample for a specific location.

Further, the processor 202 may adjust the chemical composition data for at least the first chemical compound of the first liquid stream 108 iteratively unit the TDS data of the first liquid stream 108 may be within the second threshold range. In an embodiment, the second threshold range may include at least a minimum TDS value of the first liquid stream 108 and the maximum TDS value of the first liquid stream. The minimum TDS value and the maximum TDS value may be determined based on TDS data of the reference liquid stream. In an exemplary embodiment, the TDS data of the reference liquid stream that may be calculated by the CTD device may be, for example, 35,000 mg/l. The minimum TDS value associated with the second threshold range may be, for example, 33,250 mg/l. Further, the second threshold range that may include the maximum TDS value of the first liquid stream may be, for example, 36,750 mg/l.

At 406, the processor 202 may be configured to adjust the chemical composition data of the first liquid stream 108. The processor 202 may adjust the chemical composition data of the first liquid stream 108 unit the TDS data of the first liquid stream 108 may be within the second threshold range.

In an exemplary embodiment, the processor 202 may be configured to adjust the chemical composition data of the first chemical compound, for example, chloride ion in the first liquid stream 108. Upon adjustment of the chloride ion, the TDS data of the first liquid stream may reach up to, for example, within +5% of the TDS data of the reference liquid stream and −5% of the TDS data of the reference liquid stream.

In an exemplary embodiment, the processor 202 may be further configured to perform a three-stage adjustment process to adjust the TDS data of the first liquid stream 108. The adjustments may be made to correct the TDS data of the first liquid stream 108 until it may be within the second threshold range. The second threshold range may be determined based on the TDS data of the reference liquid stream. The TDS data of the reference liquid stream may be measured from the CTD device that may be integrated with the system 102. The TDS data of the first liquid stream 108 may be the sum of ions (2 Ions) of the first liquid stream. The CTD device may be the electronic device used to detect how the conductivity and temperature of water change relative to depth. The electronic devices that may be used in the CTD device may be, for example, but not limited to, a sensor for conductivity, a sensor for temperature, and a sensor for pressure.

In an exemplary embodiment, in the first stage, the processor 202 may adjust the chemical composition data for at least the first chemical compound of the set of chemical compounds of the first liquid stream 108. In one example, the at least first chemical compound may be one of, for example, sodium (Na⁺) ions or sulphate (SO₄²⁻) ions. In another example, the processor 202 may adjust chemical composition data for at least the first chemical compound and a second chemical compound of the set of chemical compounds of the first liquid stream 108 such that the first chemical compound and the second chemical compound may be, for example, Na⁺ ions and SO₄²⁻ ions, respectively. The TDS data of the reference stream may represent the total concentration of dissolved solids in the reference stream. The processor 202 may adjust the chemical composition data of Na⁺ and SO₄² ions in the first liquid stream 108 to bring the TDS data of the first liquid stream 108 within the second threshold range. The second threshold range may be determined based on the TDS data of the reference liquid stream.

Further, upon adjusting the chemical composition data of Na⁺ ions and SO₄² ions in the first liquid stream 108, the processor 202 may be configured to determine the TDS data of the first liquid stream 108. Based on the determination, if the TDS data of the first liquid stream 108 may be within the second threshold range, the processor 202 may be configured to control the output of the first liquid stream 108.

Alternatively, based on the determination, that the TDS data of the first liquid stream 108 may not be within the second threshold range, the processor 202 may be configured to perform the second stage adjustment. In the second stage, the processor 202 may be configured to adjust the chemical composition data for a third chemical compound that may be, for example, magnesium (Mg²⁺).

Further, the processor 202 may determine the TDS data of the first liquid stream 108 after adjusting the chemical composition data of the Mg²⁺ ions. Based on the determination, if the TDS data of the first liquid stream 108 may be within the second threshold range, the processor 202 may control the output of the first liquid stream 412. The output of the first liquid stream may be, for example, input for the reverse osmosis simulation system.

Based on the determination that the TDS data of the first liquid stream 108 may not be within the second threshold range after adjusting the chemical composition data of the Mg²⁺, the processor 202 may be configured to perform a third stage.

In the third stage, the processor 202 may adjust the chemical composition data for a fourth chemical compound of the set of chemical compounds of the first liquid stream. In one example, the fourth chemical compound may be at least one of, for example, Ca²⁺ ions or K⁺ ions. In another example, the processor 202 may adjust chemical composition data for the fourth chemical compound and a fifth chemical compound of the set of chemical compounds of the first liquid stream 108 such that the fourth chemical compound and the fifth chemical compound may be, for example, calcium (Ca²⁺) ions and potassium (K⁺) ions.

Further at 410, the processor 202 may determine the TDS data of the first liquid stream 108 after adjusting the chemical composition of the Ca²⁺ ions and the K⁺ ions. Based on the determination, if the TDS data of the first liquid stream 108 may be within the second threshold range, the processor 202 may control the output of the first liquid stream 412.

In an embodiment, each of the three-stage adjustments may be iterated through a loop with all conditions stated independently. The three-stage adjustment may be implemented sequentially for TDS data (Σ Ions) adjustment of the first liquid stream. In case the TDS of the first liquid stream 108 may be within the second threshold range after the first stage adjustment, the processor 202 may not perform the second stage adjustment and the third stage adjustment.

In an exemplary embodiment, once the TDS data (ΣIons) of the first liquid stream 108 is adjusted, the TDS data (ΣIons) of the first liquid stream 108 may be compared to the TDS data of the reference liquid stream obtained from the CTD device, the processor 202 may determine whether the difference may be within the tolerance limit. The comparison may determine whether or not the charge balance of the first liquid stream 108 is 0.00%. The charge balance may indicate that the sum of positive charges of the chemical compounds of the first liquid stream 108 may be equal to the sum of negative charges of the chemical compounds of the first liquid stream 108. Further, the charge balance may maintain the overall neutrality in the first liquid stream 108. The system 102 may be configured to adjust the chemical composition of ions of the first liquid stream 108 until the charge balance of the first liquid stream 108 may be 0.00% or may approach to 0.00%.

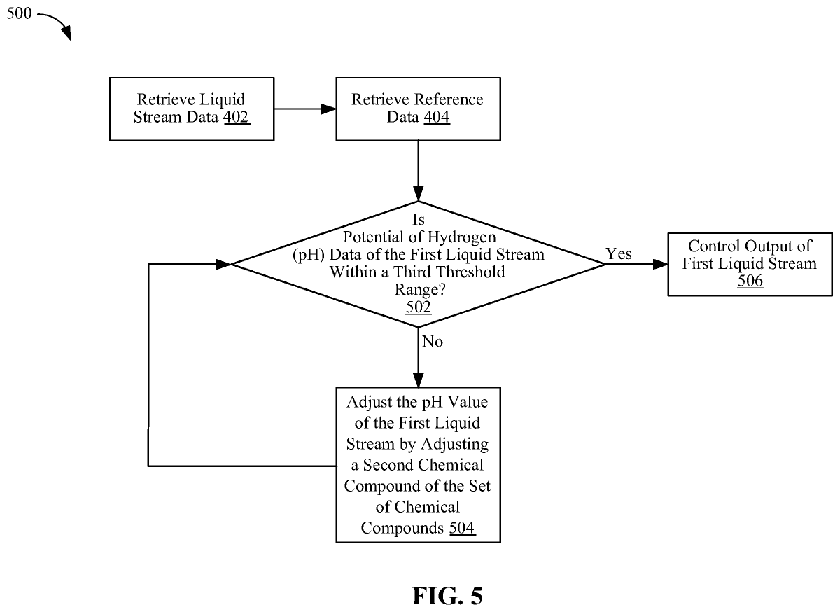

FIG. 5 illustrates exemplary operations for adjustment of the pH for normalization of seawater, in accordance with an embodiment of the disclosure. FIG. 5 is explained in conjunction with elements of FIG. 1, FIG. 2, FIG. 3, and FIG. 4. In FIG. 5, there is shown a block diagram 500 of operations performed by the system 102 for normalization of seawater.

At 502, the processor 202 may be configured to determine whether the pH data of the first liquid stream is within the third threshold range. In an embodiment, the processor 202 may adjust the chemical composition data for a second chemical compound of the set of chemical compounds of the first liquid stream 108 iteratively until the pH data of the first liquid stream 108 may be within the third threshold range.

In an exemplary embodiment, the third threshold range may include at least a minimum pH value of the first liquid stream and a maximum pH value of the first liquid stream. The minimum pH value and the maximum pH value are determined based on the pH data of the reference liquid stream. In an embodiment, the pH value may be a measure of how acidic/basic water may be. The range may lie between, for example, 0 to 14, with 7 being neutral. The pH value of less than 7 indicates acidity, whereas a pH value greater than 7 indicates base. The pH value of water may be an important measurement concerning water quality.

In an embodiment, the processor 202 may be further configured to control the output of the first liquid stream 108 based on a determination that the chemical composition data for each of the set of chemical compounds of the first liquid stream 108 may be less than the minimum chemical composition value for a corresponding chemical compound, or greater than the maximum chemical composition value for the corresponding chemical compound. The chemical composition data of the chemical compounds of the first liquid stream 108 may be determined. Based on the determination the processor 202 may output the first liquid stream 108 to adjust carbonate species in the first liquid stream 108.

At 504, the processor 202 may adjust the second chemical compound that may be, for example, carbonate species of the first liquid stream. The carbonate species may be adjusted such that the calculated pH data of the first liquid stream 108 approaches the pH data of the reference liquid stream. The processor 202 may control the reduction of the carbonate species of the first liquid stream 108 based on the determined pH data of the first liquid stream 108 being greater than the third threshold range.

In another exemplary embodiment, the processor 202 may control the addition of the carbonate species in the first liquid stream 108 based on the determined pH data of the liquid stream being lower than the third threshold range. Upon addition of the carbonate species in the first liquid stream, the carbonate species may be limited to a maximum chemical value equal to an average value of the pH data of the reference liquid stream and the minimum chemical composition value of, for example, zero.

At 506, the processor 202 may be configured to output the first liquid stream 108. The output of the first liquid stream 108 may be further analyzed by the system 102 to obtain a calculated total alkalinity (A_{T}).

In an exemplary embodiment, the processor 202 may further adjust the carbonate species of the first liquid stream 108 to obtain the calculated total alkalinity (A_{T}). Total alkalinity (A_{T}) may be indicative of a measure of the first liquid stream 108 ability to neutralize acids. Alkaline chemical compounds that may be present in the first liquid stream, for example, but not limited to, hydroxides and carbonates, may eliminate H+ ions from the first liquid stream 108, the elimination of H+ ions may reduce the acidity of the first liquid stream 108 and may further result in a higher pH value.

In an exemplary embodiment, the A_{T} may be calculated in mg/L of the chemical compound of the set of chemical compounds of the first liquid stream 108 using a three-main buffering system species. The chemical compounds of the set of chemical compounds may be, for example, calcium carbonate (CaCO₃). The three main buffering species may be, but not limited to, carbonate, and boron. Further, the processor 202 may be configured to determine a total boron concentration using pH data of the first liquid stream 108, TDS data of the first liquid stream 108, and sample temperature. The total boron concentration may be the sum of the boron species that may be present in the first liquid stream 108. The concentration of boron species may affect the chemical equilibrium of the first liquid stream 108 such as, but not limited to, the carbonate system, and pH of the first liquid stream. The boron concentration in the first liquid stream 108 may influence the carbonate system, which may further influence the alkalinity of the first liquid stream 108.

Based on the determination of the total boron concentration, the processor 202 may increase the carbonate if the determined A_{T} is less than a reference A_{T}. The reference A_{T} may be the total alkalinity that may be determined by the processor 202 by controlling a titration process of the reference liquid stream. The processor 202 may reduce the carbonate if the determined A_{T} may be greater than a reference A_{T}.

In an embodiment, upon adjusting the total alkalinity A_{T} of the first liquid stream 108, the processor 202 may be further configured to determine the charge balance error of the first liquid stream 108. Adjusting the A_{T} of the first liquid stream may involve the addition of carbonates to the first liquid stream 108. The addition of carbonates may alter the distribution of existing chemical compounds in the first liquid stream 108. Further, determining the charge balance error may ensure the accuracy of the adjustments made in the total alkalinity A_{T} of the first liquid stream. The desired outcome for the charge balance error determined by the processor 202 may be, for example, but not limited to 0.00%

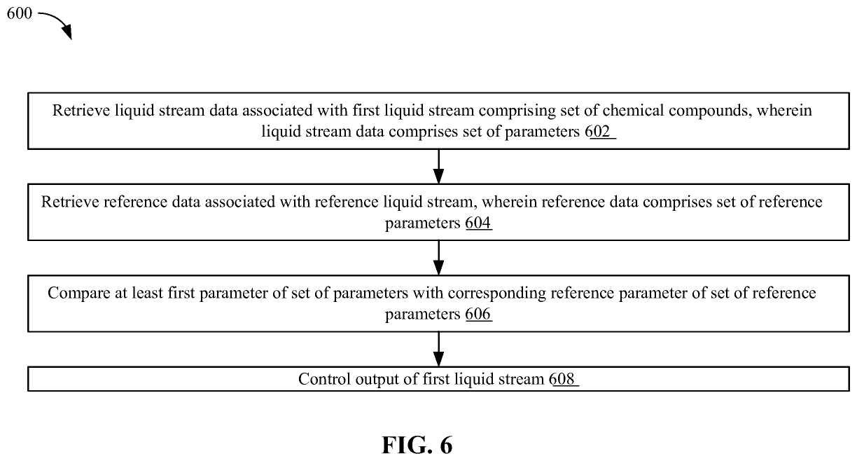

FIG. 6 is a flowchart that illustrates an exemplary method for seawater normalization, in accordance with an embodiment of the disclosure. FIG. 6 is explained in conjunction with elements from FIGS. 1, 2, 3, 4, and 5. With reference to FIG. 6, there is shown a flowchart 600. The operations of the exemplary method may be executed by any computing system, for example, by the system 102 of FIG. 1 or the processor 202 of FIG. 2. The operations of the flowchart 600 may start at 602.

At 602, liquid stream data 104 associated with the first liquid stream including a set of chemical compounds may be retrieved. The liquid stream data 104 may include a set of parameters. In an embodiment, the processor 202 may be configured to retrieve liquid stream data 104 associated with the first liquid stream 108 that may include a set of chemical compounds. The liquid stream data 104 may include the set of parameters.

At 604, reference data 106 associated with the reference liquid stream may be retrieved. The reference data 106 may include the set of reference parameters. In an embodiment, the processor 202 may be configured to retrieve the reference data 106 associated with the reference liquid stream. The reference data 106 may include the set of reference parameters.

At 606, at least the first parameter of the set of parameters may be compared with the corresponding reference parameter of the set of reference parameters. In an embodiment, the processor 202 may be configured to compare at least the first parameter of the set of parameters with the corresponding reference parameter of the set of reference parameters.

At 608, the output of the first liquid stream 108 may be controlled. In an embodiment, the processor 202 may be configured to control the output of the first liquid stream.

Alternatively, the system 102 may include means for performing each of the operations described above. In this regard, according to an example embodiment, examples of means for performing operations may comprise, for example, the processor and/or a device or circuit for executing instructions or executing an algorithm for processing information as described above.

Various embodiments of the disclosure may provide a non-transitory computer-readable medium having stored thereon computer executable instructions, which when executed by one or more processors (such as the processor 202), cause the one or more processors to carry out operations to operate a system (e.g., the system 102) for normalization of seawater. The instructions may cause the machine and/or computer to perform operations including, retrieving liquid stream data 104 associated with a first liquid stream 108 including a set of chemical compounds. The liquid stream data 104 may include a set of parameters. the operations may further include retrieving reference data 106 associated with a reference liquid stream. The reference data 106 may include a set of reference parameters. The operations may further include comparing at least the first parameter of the set of parameters with the corresponding reference parameter of the set of reference parameters. Furthermore, the operations may include controlling the output of the first liquid stream 108.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of reactants and/or functions, it should be appreciated that different combinations of reactants and/or functions may be provided by alternative embodiments without departing from the scope of the appended claims. In this regard, for example, different combinations of reactants and/or functions than those explicitly described above are also contemplated as may be set forth in some of the appended claims. Although specific terms are employed herein, they are used in a generic and descriptive sense only and not for purposes of limitation.

What is claimed:

1. A system, comprising:
a memory configured to store a computer-executable instruction; and
one or more processors coupled to the memory, wherein the one or more processors are configured to execute the computer-executable instructions that cause the one or more processors to:
retrieve liquid stream data associated with a first liquid stream comprising a set of chemical compounds, wherein the liquid stream data comprises a set of parameters;
retrieve reference data associated with a reference liquid stream, wherein the reference data comprises a set of reference parameters;
compare at least a first parameter of the set of parameters with a corresponding reference parameter of the set of reference parameters; and
control an output of the first liquid stream based on the comparison.

2. The system of claim 1, wherein the set of parameters are associated with at least one of: a chemical composition of each of the set of chemical compounds in the first liquid stream, a potential of hydrogen (pH) of the first liquid stream, a total dissolved solid (TDS) of the first liquid stream, a charge balance of the set of chemical compounds in the first liquid stream, one or more water quality parameters of the first liquid stream, and a chloride ion ratio of the first liquid stream.

3. The system of claim 1, wherein the set of reference parameters are associated with at least one of: a chemical composition for each of the set of chemical compounds in the reference liquid stream, a pH of the reference liquid stream, a TDS of the reference liquid stream, a charge balance of the set of chemical compounds in the reference liquid stream, one or more water quality parameters of the reference liquid stream, and a chloride ion ratio of the reference liquid stream.

4. The system of claim 1, wherein the one or more processors are further configured to determine whether chemical composition data for each of the set of chemical compounds of the first liquid stream is within a first threshold range, wherein the first threshold range comprises at least: a minimum chemical composition value for a corresponding chemical compound, and a maximum chemical composition value for the corresponding chemical compound, and
wherein the minimum chemical composition value and the maximum chemical composition value are determined based on chemical composition data for each of the set of chemical compounds of the reference liquid stream.

5. The system of claim 4, wherein the one or more processors are further configured to control the output of the first liquid stream based on a determination the chemical composition data for each of the set of chemical compounds of the first liquid stream is within the first threshold range, wherein the output of the first liquid stream corresponds to adjust the chemical composition data for at least a first chemical compound of the set of chemical compounds of the first liquid stream iteratively until a TDS data of the first liquid stream is within a second threshold range.

6. The system of claim 5, wherein the second threshold range comprises at least: a minimum TDS value of the first liquid stream, and a maximum TDS value of the first liquid stream, and wherein the minimum TDS value and the maximum TDS value are determined based on TDS data of the reference liquid stream.

7. The system of claim 4, wherein the one or more processors are further configured to control the output of the first liquid stream based on a determination that the chemical composition data for each of the set of chemical compounds of the first liquid stream is less than the minimum chemical composition value for a corresponding chemical compound, or greater than the maximum chemical composition value for the corresponding chemical compound, and
wherein the output of the first liquid stream corresponds to an adjustment of the chemical composition data for a second chemical compound of the set of chemical compounds of the first liquid stream iteratively until pH data of the first liquid stream is within a third threshold range.

8. The system of claim 7, wherein the third threshold range comprises at least: a minimum pH value of the first liquid stream, and a maximum pH value of the first liquid stream, and wherein the minimum pH value and the maximum pH value are determined based on pH data of the reference liquid stream.

9. A method, comprising:
retrieving liquid stream data associated with a first liquid stream comprising a set of chemical compounds, wherein the liquid stream data comprises a set of parameters;
retrieving reference data associated with a reference liquid stream, wherein the reference data comprises a set of reference parameters;
comparing at least the first parameter of the set of parameters with the corresponding reference parameter of the set of reference parameters; and
controlling an output of the first liquid stream based on the comparison.

10. The method of claim 9, wherein the set of parameters are associated with at least one of: a chemical composition or each of the set of chemical compounds in the first liquid stream, a potential of hydrogen (pH) of the first liquid stream, a total dissolved solid (TDS) of the first liquid stream, a charge balance of the set of chemical compounds in the first liquid stream, one or more water quality parameters of the first liquid stream, and a chloride ion ratio of the first liquid stream.

11. The method of claim 9, wherein the set of reference parameters are associated with at least one of: a chemical composition for each of the set of chemical compounds in a reference liquid stream, a pH of the reference liquid stream, a TDS of the reference liquid stream, a charge balance of the set of chemical compounds in the reference liquid stream, one or more water quality parameters of the reference liquid stream, and a chloride ion ratio of the reference liquid stream.

12. The method of claim 9, further comprising determining whether chemical composition data for each of the set of chemical compounds of the first liquid stream is within a first threshold range, wherein the first threshold range comprises at least: a minimum chemical composition value for a corresponding chemical compound, and a maximum chemical composition value for the corresponding chemical compound, and
wherein the minimum chemical composition value and the maximum chemical composition value are determined based on chemical composition data for each of the set of chemical compounds of the reference liquid stream.

13. The method of claim 12, further comprising controlling the output of the first liquid stream based on a determination the chemical composition data for each of the set of chemical compounds of the first liquid stream is within the first threshold range, wherein the output of the first liquid stream corresponds to adjust the chemical composition data for at least a first chemical compound of the set of chemical compounds of the first liquid stream iteratively until a TDS data of the first liquid stream is within a second threshold range.

14. The method of claim 13, wherein the second threshold range comprises at least: a minimum TDS value of the first liquid stream, and a maximum TDS value of the first liquid stream, and wherein the minimum TDS value and the maximum TDS value are determined based on TDS data of the reference liquid stream.

15. The method of claim 12, further comprising controlling the output of the first liquid stream based on a determination that the chemical composition data for each of the set of chemical compounds of the first liquid stream is less than the minimum chemical composition value for a corresponding chemical compound, or greater than the maximum chemical composition value for the corresponding chemical compound, and
wherein the output of the first liquid stream corresponds to an adjustment of the chemical composition data for a second chemical compound of the set of chemical compounds of the first liquid stream iteratively until pH data of the first liquid stream is within a third threshold range.

16. The method of claim 15, wherein the third threshold range comprises at least: a minimum pH value of the first liquid stream, and a maximum pH value of the first liquid stream, and wherein the minimum pH value and the maximum pH value are determined based on pH data of the reference liquid stream.

17. A computer programmable product comprising a non-transitory computer readable medium having stored thereon computer executable instructions, which when executed by one or more processors, cause the one or more processors to carry out operations comprising:
retrieving liquid stream data associated with a first liquid stream comprising a set of chemical compounds, wherein the liquid stream data comprises a set of parameters;
retrieving reference data associated with a reference liquid stream, wherein the reference data comprises a set of reference parameters;
comparing at least a first parameter of the set of parameters with a corresponding reference parameter of the set of reference parameters; and
controlling an output of the first liquid stream based on the comparison.

18. The computer programmable product of claim 17, wherein the set of parameters are associated with at least one of: a chemical composition or each of the set of chemical compounds in the first liquid stream, a potential of hydrogen (pH) of the first liquid stream, a total dissolved solids (TDS) of the first liquid stream, a charge balance of the set of chemical compounds in the first liquid stream, one or more water quality parameters of the first liquid stream, and a chloride ion ratio of the first liquid stream.

19. The computer programmable product of claim 17, wherein the set of reference parameters are associated with at least one of: a chemical composition for each of the set of chemical compounds in a reference liquid stream, a pH of the reference liquid stream, a TDS of the reference liquid stream, a charge balance of the set of chemical compounds in the reference liquid stream, one or more water quality parameters of the reference liquid stream, and a chloride ion ratio of the reference liquid stream.

20. The computer programmable product of claim 17, further comprising determining whether chemical composition data for each of the set of chemical compounds of the first liquid stream is within a first threshold range, wherein the first threshold range comprises at least: a minimum chemical composition value for a corresponding chemical compound, and a maximum chemical composition value for the corresponding chemical compound, and
wherein the minimum chemical composition value and the maximum chemical composition value are determined based on chemical composition data for each of the set of chemical compounds of the reference liquid stream.
